SEQUENCE LISTING (18 sequences)

SEQ ID NO: 1 - DNA, 20 bp, artificial sequence
  pGL2 G quartet motif sequence
gggatacgacaaggatatgg

SEQ ID NO: 2 - DNA, 20 bp, artificial sequence
  pGL3 G quartet motif sequence
ggtatcaggcaaggatatgg

SEQ ID NO: 3 - DNA, 24 bp, artificial sequence
  G quartet motif sequence
wggnnnnwggnnnnwggnnnnwgg

SEQ ID NO: 4 - DNA, 24 bp, artificial sequence
  G quartet motif sequence
nggnnnnnggnnnnnggnnnnngg

SEQ ID NO: 5 - DNA, 36 bp, artificial sequence
  primer
ccatctgccaggtatcagacaaggatatgggctcac

SEQ ID NO: 6 - DNA, 36 bp, artificial sequence
  primer
gtgagcccatatccttgtctgatacctggcagatgg

SEQ ID NO: 7 - DNA, 174 bp, artificial sequence
  plasmid
cacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttcca

SEQ ID NO: 8 - DNA, 174 bp, artificial sequence
  plasmid
cacgaaattgcttctggtggcgctcccctctctaaggaagtcggggaagcggttgccaagaggttccatctgccaggtatcagacaaggatatgggctcactgagactacatcagctattctgattacacccgagggggatgataaaccgggcgcggtcggtaaagttgttcca

SEQ ID NO: 9 - DNA, 31 bp, artificial sequence
  primer
tgctctagacacgaaattgcttctggtggcc

SEQ ID NO: 10 - DNA, 29 bp, artificial sequence
  primer
tgctctagatggaacaactttaccgcgcc

SEQ ID NO: 11 - DNA, 29 bp, artificial sequence
  primer
ccgctcgaggccaagaggttccatctgcc

SEQ ID NO: 12 - DNA, 32 bp, artificial sequence
  primer
ccaagcttttagctgatgtagtctcagtgagc

SEQ ID NO: 13 - DNA, 26 bp, artificial sequence
  annealing oligonucleotide
agcttggtatcaggcaaggatatggg

SEQ ID NO: 14 - DNA, 26 bp, artificial sequence
  annealing oligonucleotide
aattcccatatccttgcctgatacca

SEQ ID NO: 15 - DNA, 25 bp, artificial sequence
  annealing oligonucleotide
aattcggatcaggcaaggatatggg

SEQ ID NO: 16 - DNA, 26 bp, artificial sequence
  annealing oligonucleotide
tcgacccatatccttgcctgataccg

SEQ ID NO: 17 - DNA, 20 bp, artificial sequence
  primer
ctacctgagcacccagtccg

SEQ ID NO: 18 - DNA, 18 bp, artificial sequence
  primer
tgatcgcgcttctcgttg

(19) United States
(12) Patent Application Publication
(10) Pub. No.: US 2012/0208233 A1
(43) Pub. Date: Aug. 16, 2012

(54) ENHANCEMENT OF PROTEIN PRODUCTION IN EUKARYOTIC CELLS

(75) Inventors: Susann Schweiger, Dundee (GB); Beatriz Aranda-Orgillés, New York, NY (US); Rainer Schneider, Worgl (AT); Stefan Roepcke, Konstanz (DE); Sven Krause, Gottingen (DE)

(73) Assignee: Texas Department of Transportation

(21) Appl. No.: 12/672,056

(22) PCT Filed: Aug. 4, 2008
(86) PCT No.: PCT/EP2008/006511

§ 371 Date: Aug. 30, 2011

(30) Foreign Application Priority Data
Aug. 3, 2007 (EP) EP07015280.6

(51) Int. Cl. C12P 21/06; C12N 15/63

(52) U.S. Cl. 435/69.1; 435/320.1

(57) ABSTRACT

An embodiment relates to the increased expression of polypeptides in eukaryotic cells. A method for increased production is described using an expression vector comprising a polynucleotide encoding a desired polypeptide and a G quartet like motif sequence.

### BACKGROUND OF THE INVENTION

1. Field of the Invention

The present invention relates to the increased expression of polypeptides in eukaryotic cells. A method for increased production is described using an expression vector comprising a polynucleotide encoding a desired polypeptide and a G quartet like motif sequence.

2. Description of the Relevant Art

The production of eukaryote proteins in cell systems is an important issue for a wide range of applications in modern biotechnology. Applications range from research into the biological function of proteins to their production as biopharmaceuticals and diagnostic reagents and the development of transgenic animals and plants. However importantly, most of these applications require correct post-translational modification. Production of eukaryote proteins in prokaryotes is limited by the lack of post-translational processing, including appropriate protein folding and secondary modifications such as glycosylation and some phosphorylations. Also, the absence of most cell organdies prohibits the functional analysis of eukaryotic proteins in prokaryotes. Thus many applications, including gene therapy, the fabrication of recombinant protein therapeutics and diagnostics and the production of proteins as well as the production of transgenic plants and animals for in vivo and in vitro studies, depend on proteins produced in eukaryotic cells. Unfortunately, the relatively low productivity of most eukaryotic cell systems is the most significant hindrance.

Efforts have been undertaken during the last few years to improve productivity of eukaryotic protein expression systems. The site of integration of a plasmid into the genome of an acceptor cell line has a major impact on its transcription, for example, integration into heterochromatin results in little or no expression. Several strategies have been developed to overcome this problem. Targeted integration via homologous recombination is one possibility and enzymes with recombinase activity, such as bacteriophage P1 Cre recombinase, lambda phage integrase or yeast Flp recombinase, can be used to enhance the probability of targeted integration. Also due to the strong influence of splicing on translation most modern expression vectors include an intron between the promoter and the cDNA coding sequence. Additionally, engineering of rare tRNA codons into more frequently used tRNA codons encoding identical amino acids, can be used to convert a gene expressed at low levels into a high-expressing gene. In another strategy host cells have been genetically modified into higher efficiency producers by integrating genes into their genome that make them resistant to influences that reduce viability or growth-promoting proto-oncogenes, cell cycle control genes, growth factors and anti-apoptotic genes.

Guanine quartet sequences and guanine quadruplexes (G-quartet like sequences) are known in the art. For example, Bonnal et al., (2003) Journal of Biological Chemistry, Vol. 278(41) pp. 39330-39336 discloses an expression vector having a internal G-quartet like sequence; WO 99/14346 and WO 2006/022712 disclose G-quartet like sequences and that these sequences are involved in regulation of gene expression; and Aranda-Orgilles, B. (2006) Online URL: http://www.diss.fu-berlin.de/2006/660/indexe.html> discloses the interaction between the MID1 protein and the α4 protein and a G-quartet like sequence. However none of these documents disclose that a G-quartet like sequence can be used in a method to increase protein production in eukaryotic cells as disclosed in the present application.

### SUMMARY OF THE INVENTION

It has been surprisingly found that protein production is increased by polypeptide translation from and/or increased mRNA stability of an mRNA carrying G-quartet like sequences. A method and expression vector that optimizes the productivity of eukaryotic expression systems at the translation level is described herein.

In one embodiment, a method for increasing production of a polypeptide in a eukaryote host cell includes: transforming the eukaryote host cell with an expression vector comprising a polynucleotide sequence that encodes an open reading frame for the polypeptide and at least one G quartet motif sequence; culturing the transformed cells; and isolating the expressed polypeptide. In some embodiments, the amount of the polypeptide produced is increased as the number of G quartet motif sequences is increased.

In an embodiment, the number of G quartet motif sequences is between 1 and 5. In the expression vector the G quartet motif sequence may be situated 3′ of the polypeptide open reading frame. In another embodiment, the expression vector the G quartet motif sequence may be situated within the polypeptide open reading frame. The G quartet motif sequence, in an embodiment, encodes an RNA consensus sequence of:

(SEQ ID NO: 3)
WGGN₍₁₋₄₎WGGN₍₁₋₄₎WGGN₍₁₋₄₎WGG.

The polypeptide may be a human polypeptide, an animal polypeptide, a plant polypeptide, or a recombinant polypeptide.

In an embodiment, the eukaryote cell is further transformed with an expression vector encoding the MID1 protein. In an embodiment, the eukaryote cell is further transformed with an expression vector encoding the α4 protein.

A G quartet motif sequence, in an embodiment, is used to increase production of a polypeptide in a eukaryote host cell, wherein the eukaryote cell is transformed with an expression vector comprising a polynucleotide sequence that encodes an open reading frame for the polypeptide and at least one G quartet motif sequence.

In another embodiment, an expression vector includes a polynucleotide encoding an open reading frame for a polypeptide and at least one G quartet motif sequence, wherein the G quartet motif sequence increases production of the polypeptide in a eukaryote host cell.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1: EFNB1 mRNAs either lacking the entire 3′UTR (−3′U) or including one G-quartet (+1G), two G-quartets (+2G), three G-quartets (+3G) or four G-quartets (+4G), with antisense transcripts of 3 G-quartet (+3G-AS) and four G-quartet (+4G-AS) mRNAs used as background controls. G-quartets were amplified and in vitro transcribed using biotinylated ribonucleotides. Elution fractions of RNA-protein pull-down assays in FLAG-MID1 over-expressing cells were analysed for the presence of FLAG-MID1 with an anti-FLAG antibody.

FIG. 2: pGL3 had 6 times higher luciferase activity from the firefly luciferase gene compared to pGL2 as measured in relative light units.

FIG. 3: Basal activities of the wild-type pGL3 (GL3) and a pGL3 vector mutated at a core guanine of the predicted G-quartet (GL3mut) as measured in relative light units.

FIG. 4: Induction of luciferase activity is observed by co-transfection with wild-type MID1, but not with a mutated (A130T) form of MID1. Induction is seen of the pGL3 wild-type firefly luciferase gene (a, b) but not of a firefly luciferase gene with a mutated G-quartet sequence motif (GL3mut) or the pGL2 firefly luciferase gene.

4a. Firefly luciferase activity recorded from HeLa cells transfected with vectors either containing (GL3) or lacking (GL2) a functional MIDAS G-quartet sequence motif, and co-transfected with empty pCMV vector (-cmv), wild-type-MID1 (-MID1), or mutated, inactive MID1 (-A130T).

4b. Firefly luciferase activity recorded from HeLa cells transfected solely with pGL3 vectors containing either a functional MIDAS G-quartet sequence motif (GL3) or a mutated MIDAS G-quartet sequence motif (GL3mut) within the open reading frame, and also co-expressing either empty pCMV vector (+cmv), wild-t e-MID1 (+MID1), or mutated, dysfunctional MID1 (+A130T).

FIG. 5: FLAG-MID1 was identified in the elution fraction of an RNA-protein pulldown assay performed with biotinylated firefly luciferase RNA in-vitro transcribed from the pGL3 vector (a, b), but not in an assay performed with firefly RNA transcribed from pGL2 (a), with renilla luciferase RNA (a, b) or in an experiment without RNA. Only little binding was observed with RNA transcribed from a pGL3 vector carrying a mutation in the G-quartet structure (b).

5a. RNA-protein pulldown assay: Lysates from FLAG-MID1-overexpressing HeLa cells were incubated with biotinylated RNA in vitro-transcribed from either pGL3 (Firefly-luc+pGL3) or pGL2 (Firefly-luc pGL2) vectors, or from a renilla vector without MIDAS G-quartet sequence motif (renilla pRL). Binding fractions and lysate were analyzed on a Western blot with an anti-FLAG antibody.

5b. RNA-protein pulldown assay: Lysates from FLAG-MID1 overexpressing HeLa cells were incubated alone (No RNA) or with biotinylated RNA in vitro-transcribed from a pGL3 vector containing either a functional MIDAS G-quartet sequence motif (Firefly) or a mutated MIDAS G-quartet sequence motif (Mutated), or from a renilla vector lacking the MIDAS motif (renilla). Binding fractions and lysate were analysed on a Western blot with an anti-FLAG antibody.

FIG. 6: MIDAS G-quartet sequence motif sequence increases production of proteins in vitro and in vivo.

6a. In vitro translation of a firefly luciferase gene containing either a functional MIDAS G-quartet sequence motif (GL3) or a mutated MIDAS G-quartet sequence motif (GL3m) motif. Luciferase activities are shown.

6b. Luciferase activity in CHO cells expressing a mutated pGL3 vector (GL3m) carrying a functional (GL3m-MIDAS) or a non-functional (GL3m-MIDASm) MIDAS G-quartet sequence motif 3′ to the firefly luciferase coding region.

6c. Intensities of green fluorescence recorded from CHO cells transfected with GFP-encoding vectors (pEGFP) either containing (MIDAS) or lacking (empty) a MIDAS G-quartet sequence motif 3′ to the coding region for the GFP protein, measured by FACS.

6d. Production of GFP from pEGFP vectors with no (empty), single (MIDAS), double (2×MIDAS) or triple (3×MIDAS) functional MIDAS G-quartet sequence motif within the 3′UTR of the GFP gene in CHO cells. Fluorescence intensities measured by FACS.

FIG. 7: Production of GFP from pIRES-vectors either containing (GFPi-MIDAS) or lacking (GFPi) a functional MIDAS G-quartet sequence motif 3′ to the coding region of the GFP protein, in 7a) HeLa, 7b) HEK, 7c) COS7 and 7d) CHO cells. Fluorescence intensities measured by FACS (top panels), GFP RNA levels relative to those of GAPDH, measured by real-time PCR (centre panels), and ratios between fluorescence intensities and mRNA expression (translation efficiencies, bottom panels) are shown.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

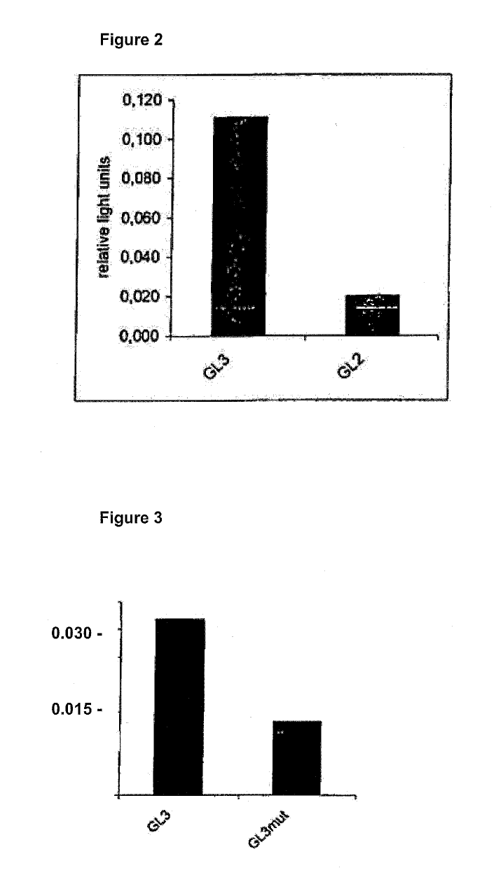

The present inventors have identified a cytoskeleton-associated ribosomal complex that includes active polyribosomes and components of the translation-controlling mTOR (mammalian target of rapamycin) pathway, such as the MID1 and α4 proteins, which is present in all cell types tested, including yeast and insect cells. mRNA associated to this complex is stabilized, thus significantly increasing their translation. Furthermore, the present inventors found that mRNA containing G-quartet like RNA motifs having a consensus sequence of WGG-N(1-4)-WGG-N(1-4)-WGG-N(1-4)-WGG has a high affinity to the described ribosome/mTOR MID1 complex. Thus the MID1 complex associates with mRNAs via a sequence motif, which we called MIDAS (MID1 association sequence) G-quartet sequence motif, that corresponds to the consensus sequence above and this association enhances the translation efficiency of these mRNAs several fold as demonstrated in Examples 2 to 6 and FIGS. 2 to 5 and FIG. 6d. Furthermore, the observed effects of the MID1/α4 protein complex on the synthesis of proteins encoded by RNAs with MIDAS G-quartet sequence motifs has multiple implications for the production of proteins in eukaryotic cell systems that either rely on high productivity or on dose-dependent protein expression.

G-quartet like RNA motifs have been described previously, however the biological function of these structures is still under investigation. They have been variously associated with mRNA turnover and HIV packaging, as well as being proposed to have a regulatory role in cell metabolism.

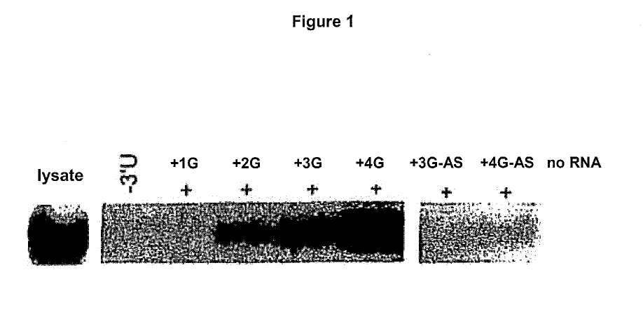

The present inventors have demonstrated that the MIDAS G-quartet like motifs having a consensus sequence of WGG-N(1-4)-WGG-N(1-4)-WGG-N(1-4)-WGG (SEQ ID NO: 3) mediate and stabilize the binding of mRNAs carrying the G-quartet like sequence to the ribosome complex. Further the present inventors have demonstrated that binding is increased in a linear additive manner by the presence of the MIDAS G-quartet like motif sequences. As described in Example 1 and shown in FIG. 1 and Example 6 and FIG. 6d as the number of MIDAS G-quartet like motif sequences in an mRNA was increased up to four MIDAS G-quartet like motifs, the amount of the protein production from the mRNA also increased.

The position of the MIDAS G-quartet like motif(s) within the respective mRNA does not seem to play a role, meaning that the MIDAS G-quartet like motif sequence can be located inside or outside (3′ or 5′) of an open reading frame. When the positioning of essential sequence MIDAS G-quartet like sequence motifs is outside of the open reading frame, this method will not influence the protein sequence or post-translational modifications, guaranteeing unaltered quality of the produced proteins.

Without wishing to be bound by any particular theory the inventors propose that the possible molecular basis of the increase in protein production is both an increase of mRNA stability mediated by proteins interacting with the complex and an induction of translation via the mTOR signalling cascade.

Production of recombinant therapeutic proteins in cultivated mammalian cells has surpassed that in microbial expression systems because of the greater capacity of eukaryotic cells for proper folding, assembly and post-translational modification of proteins, which has helped to enhance the quality and efficacy of protein products for research and clinical applications. However, the production of complex proteins in mammalian cells remains extremely costly, and consequently there is high demand for novel approaches to lower costs and enhance protein yields. Therefore our present finding that MIDAS motifs introduced into mRNAs markedly enhanced the production of desired proteins will permit considerable improvements in the cost-efficient production of sophisticated biopharmaceuticals.

We describe herein a strategy to enhance protein production by increased translation from an mRNA carrying MIDAS G-quartet like sequences 3′ or 5′ of or within an open reading frame in cell lines and transgenic plants and animals. Furthermore, protein amounts produced in the host cell lines or transgenic plants or animals can be titered upon the number of G-quartets, preferably up to five incorporated in the vector.

Preferably the MIDAS G-quartet motif encodes the following sequence:

(SEQ ID NO: 4)
NGGN₍₂₋₅₎GGN₍₂₋₅₎GGN₍₂₋₅₎GG

wherein N is any nucleotide.
Further preferably the MIDAS G quartet motif sequence encodes an RNA consensus sequence of:

(SEQ ID NO: 3)
WGGN₍₁₋₄₎WGGN₍₁₋₄₎WGGN₍₁₋₄₎WGG

wherein N is any nucleotide and W is A or T.

Preferably, if incorporated 3′ of an open reading-frame, the RNA motif enhances protein production 2-10 fold as demonstrated in Example 6 and FIGS. 6b and 6c. Further preferably co-transfection with a plasmid expressing the MID1 or α4 gene additionally increases protein production another 5-fold.

Also the protein production increases according to the increasing number of MIDAS G-quartets present and can be regulated by the number of MIDAS G-quartets incorporated in the vector as demonstrated in Example 6 and FIG. 6d. Thus the amount of polypeptide produced is increased as the number of MIDAS G quartet motif sequences is increased. For example, a larger amount of polypeptide is produced when four MIDAS G quartet motif sequences are used that when only one MIDAS G quartet motif sequence is used. Preferably up to five MIDAS G-quartet like motifs shall be used in the same vector.

As used herein “increased production of a polypeptide” is defined as that the amount of a polypeptide produced by a host cell in the presence of the MIDAS G quartet motif sequence is greater than the amount of the polypeptide produced in the absence of the MIDAS G quartet motif sequence.

As used herein “polypeptide” is defined as a polymer of amino acids joined by peptide bonds. The polypeptide is encoded by a corresponding polynucleotide sequence.

As used herein “polynucleotide sequence” is defined as a DNA sequence capable of encoding a polypeptide.

As used herein “protein” is defined as a macromolecule comprising one or more polypeptide chains. A protein may also comprise non-peptide components, such as carbohydrate groups.

As used herein “expression vector” is defined as a nucleic acid molecule encoding a polynucleotide sequence that is expressed in a host cell. The expression vector can also comprise regulatory sequences, termination sequences and sequence encoding a selection factor(s). The expression vector may be, for example, a plasmid or a viral or retroviral vector, such as the pLenti6-V5, the pLenti6/V5-DEST Gateway™, the pAd/PL-DEST™ the pAd/CMV/V5-DESTT™, the BD BaculoGold™, the AcNPV, the pCMV/Blue/OuabainR, the pCMV/OuabainR, the pRK-5, the pAPtag-4, the Gateway™ pCMV•SPORT6, the pcDNA™3.1, the pcDNA™4, the pcDNA™5, the pcDNA™6, the pCEP4, the pCMV, the pDisplay™, the pEF, the pEF1, the pEF4/, the pEF6, the Plasmid pCMV•SPORT, the pREP4, the pSecTag2, the pTracer™-EF, the pUB6, the pVAX1, the pVP22, the pZeoSV2, the pBud, the pIRES, the pCI, the pSI, the pTARGET™, the pFLAG®-MAC, the p3XFLAG-CMV™, the pBICEP-CMV™, the pcDNA, the pCMV-FLAG®, the pFLAG®-CMV, the pRc, the pGS, the pUSEamp, the pGL, the pRL, the phMGFP, the pEGFP, the pGFP, the pYFP, the pRFP, the pDSred, the pSV-β-Gal, the pCAT®3, the pHTS-MCS, the pcherry, the pHT3, the pStec, the FMHGW, the rtTA, the pHR′CMV, the pSNVRU5luc, the pSNVluc, the pTRL2, the pCALwL, the tTS, the psiRNA™, the pBROAD, the pWHERE, the pMONO, the pSELECT, the pVITRO, the pVIVO, the pFUSE-Fc and others.

As used herein “host cell” is defined as a cell capable of producing a polypeptide encoded by a polynucleotide sequence carried on an expression vector. The host cell may be, for example, a yeast cell, an insect cell, or a mammalian cell. Examples of specific host cells include the following: any insect cell line of interest, any plant cell of interest, any mammalian cells of interest or yeast or any transgenic animal cells of interest. For example, Table 1 provides a listing of possible mammalian host cells that could be used with an expression vector containing at least one G quartet motif sequence. However the listing is not exhaustive and the expression vector containing at least one MIDAS G quartet motif sequence could be used in a mammalian host cell that is not listed in Table 1. Example 7 and FIGS. 7a, 7b, 7c and 7d demonstrate the activity of the MIDAS G quartet motif sequence in a method of increasing protein production in HeLa (FIG. 7a), CHO (FIG. 7b), COS7 (FIG. 7c) and HEK293 (FIG. 7d) mammalian cell lines.

TABLE 1
Mammalian host cells

| Cell line | Cell type | Species |
|---|---|---|
| 143B | Osteosarcoma | Human |
| 15P-1 | Sertoli (Testis) | Mouse |
| 16HBE | Epithelial airway | Human |
| 293, 293H, 293F | Kidney | Human |
| 293T | Embryonic kidney | Human |
| 3T3-F442A | Adipocytes | Mouse |
| A10 | Smooth muscle | Rat |
| A20.J; M12 | Lymphoid | Mouse B cell |
| | | lymphoma |
| A498; UMRC 6 | Kidney carcinoma | Human |
| A549 | Lung | Human carcinoma |
| AKR | Spleen | Mouse |
| AR4-2J | Pancreas | Rat |
| AS52; CHO-DXB11 | Ovary | Hamster |
| AtT-20 | Pituitary | Mouse |
| B16 | Melanoma | Mouse |
| BEAS-2B | Bronchial epithelial | Human |
| | cells | |
| BL3.1 | Lymphoid | Bovine leukemia |
| Blastoderm cells | | Chicken |
| C127 | Breast Cancer | Mouse |
| C2C12 | Myoblast | Mouse |
| C2C12 | Muscle | Cow |
| C-33 A | Cervical Cancer | Human |
| C6 | Glioma | Rat |
| Capan-2 | Pancreas cancer | Human |
| CHO | Ovary | Chinese Hamster |
| | (epithelial like) | |
| CHO-K1 | Ovary | Hamster |
| COLO 320 HSR; SW40; | Colon | Human cancer |
| 3SW480; Caco-2 | | |
| COS1, COS-7 | Kidney | Green Monkey |
| CT60 | Ovary | Hamster |
| D3 | Stem cells | Mouse |
| Dede | Lung | Hamster |
| F11 | Mouse | Mouse/Rat |
| | neuroblastoma + | |
| | Rat DRG neurons | |
| FLK | Kidney | Sheep |
| FTO-2B | Liver cancer | Rat |
| GD25-β1 | Embryonal fibroblast | Mouse |
| GH3 | Pituitary tumor | Rat |
| H187; NCI-H146 | Lung | Human small cell |
| | | carcinoma |
| HBL-100 | Transformed Breast | Human |
| HCT116 | Colon | Human cancer |
| HEK293 | Embryonic kidney | Human |
| HeLa | Cervical Cancer | Human |
| Hep 3B | Liver cancer | Human |
| Hep2 | Laryngeal Epithelium | Human |
| HepG2 | Hepatoma | Human |
| Hs 578T; T-47D; | Breast Cancer | Human |
| MDA-MB-231; ZR-75-1 | | |
| HT-29 | Colon | Human cancer |
| Huh7 | Liver cancer | Human |
| Isikawa | Endometrial cancer | Human |
| Jurkat | Lymphoid | Human lymphoma |
| KG-1; KG1a; K562 | leukemia | Human |
| L | Connective Tissue | Mouse |
| L6E9 | Muscle | Rat |
| LLC | Lung | Mouse carcinoma |
| LLC-PK | Kidney | Pig |
| LNCaP; PC-3 | Prostate | Human |
| MCF-7 | Breast | Human |
| | Adenocarcinoma | |
| MCF7 | Breast Cancer | Human |
| MDCK | Kidney | Canine |
| MeWo | Melanoma | Human |
| MOLT-4 | Lymphoid | Human leukemia |
| MRC-5 | Lung | Human |
| MSN610.2; Morris 7777; | Liver cancer | Rat |
| H35; McA-RH7777 | | |
| Mv 1 Lu | Lung Fibroblasts | Mink |
| N2A | Neuroblastoma | Mouse |
| NCI-H23 | Lung | Human cancer |
| Neuro-2a | Neuroblastoma | Mouse |
| NG108-15 | Neurablastoma-glioma | Mouse-Rat |
| | fusion | |
| NIH 3T3; TA1; | Fibroblasts | Mouse |
| Y-2; NIH 3T6; 10T½ | | |
| NIH-3T3 | Fibroblasts | Mouse |
| NMU | Breast Cancer | Rat |
| P19; F9 | Embryonic carcinoma | Mouse |
| P388D1 | Lymphoid | Mouse lymphoma |
| PC 12 | Pheochromocytoma | Rat |
| Primary chondrocytes | | Chicken |
| Primary Endothelial | Endothelial | Rabbit |
| Primary fetal astrocyte | Astrocyte | Rat |
| Primary fetal islet cells | Pancreas islet | Rat |
| Primary Fibroblast | Fibroblasts | Human |
| Primary fibroblasts | | Chicken |
| Primary hematopoetic | Stem cells | Human |
| CD34+ | | |
| Primary hepatocytes | | Chicken |
| Primary HUAEC | Endothelial | Human |
| Primary HUVEC | Endothelial | Human |
| Primary keratinocytes | Skin | Human |
| Primary Liver | Liver | Dog |
| Primary Liver | Liver | Mouse |
| Primary Liver | Liver | Rat |
| Primary Ovary | Ovary | Mouse |
| Primary Pituitary | Pituitary | Rat |
| Primary; G8 | Muscle | Mouse |
| Primary; J774.16; RAW | Monocytes | Mouse macrophage |
| 264.7 | | |
| R1610 | Lung | Hamster |
| SK-BR-3 | Breast Cancer | Human |
| SK-N-MC | Neuroblastoma | Human |
| SK-N-SH | Neuroblastoma | Human |
| SKOV3 | Ovary cancer | Human |
| SK-UT-1 | Uterus | Human |
| Sperm | Sperm | Mouse |
| SW13 | Adrenal carcinoma | Human |
| Swiss 3T3 | Fibroblasts | Mouse |
| T24 | Bladder carcinoma | Human |
| T84 | Colon | Human cancer |
| THP-1 | Macrophages/ | Human monocyte |
| Trachea smooth muscle | Muscle | Cow |
| U-2 OS | Bone sarcoma | Human |
| U373 | Glioblastoma | Human |
| U-87; A-172 | Glioma | Human |
| U937 | Lymphoid | Human lymphoma |
| WI-38 | Lung | Human |

As used herein a “host” is an organism capable of producing a polypeptide encoded by a polynucleotide sequence carried on an expression vector. The host may be, for example, a transgenic animal.

One embodiment is directed to a method for increasing production of a polypeptide in a eukaryote host cell comprising transforming the eukaryote host cell with an expression vector comprising a polynucleotide sequence that encodes an open reading frame for the polypeptide and at least one and preferably up to five G quartet like motif sequences, culturing the transformed cells under suitable conditions and isolating the expressed polypeptide.

General methods for constructing expression vectors, transforming host cells, culturing host cells and isolating an expressed polypeptide are well known in the art. For example, the commercial products and accompanying protocols available from any of Invitrogen Corporation, InvivoGen, Promega Corporation, or Sigma-Aldrich Co. are suitable to carry out the embodiments described herein.

In the method the amount of a polypeptide produced in the presence of a G quartet motif sequence is greater than the amount of polypeptide produced in the absence of a G quartet motif sequence.

Preferably the number of G quartet motif sequences is between 1 and 5, more preferably between 3 and 4.

In the method the amount of the polypeptide produced is increased as the number of G quartet motif sequences is increased.

The method is suitable for use in an in vitro eukaryote expression system. The method is also suitable for use in an in vivo eukaryote expression system.

Preferably in the expression vector the G quartet motif sequence is situated 3′ of the polypeptide open reading frame. When the G quartet motif sequence is incorporated 3′ of the open reading-frame preferably protein production is increased 2-10 fold.

Alternatively in the expression vector the G quartet motif sequence is situated within the polypeptide open reading frame.

Preferably the G-quartet motif encodes the following sequence:

(SEQ ID NO: 4)
NGGN₍₂₋₅₎GGN₍₂₋₅₎GGN₍₂₋₅₎GG

wherein N is any nucleotide.

Further preferably the G quartet motif sequence encodes an RNA consensus sequence of:

(SEQ ID NO: 3)
WGGN₍₁₋₄₎WGGN₍₁₋₄₎WGGN₍₁₋₄₎WGG

wherein N is any nucleotide and W is A or T.
Further preferably the G quartet motif sequence is:

(SEQ ID NO: 2)
GGTATCAGGCAAGGATATGG

Preferably the polypeptide is a human polypeptide. Alternatively the polypeptide is an animal polypeptide. Further preferably the polypeptide is a recombinant polypeptide. Further preferably the polypeptide is a plant polypeptide.

Additionally the eukaryote cell can be further transformed with an expression vector encoding the MID1 protein. Preferably when the host cell is co-transfected with a plasmid expressing the MID1 gene protein production is increased 5-fold. The host cell can also be further transformed with an expression vector encoding the α4 protein.

Preferably the host cell is any insect cell of interest or any plant cell of interest or any mammalian cell of interest or yeast or any transgenic animal cell of interest.

An embodiment is also directed to the use of a G quartet motif sequence to increase production of a polypeptide in a eukaryote host cell, wherein the eukaryote host cell is transformed with an expression vector comprising a polynucleotide sequence that encodes an open reading frame for the polypeptide and at least one G quartet motif sequence.

In the use the amount of a polypeptide produced in the presence of a G quartet motif sequence is greater than the amount of polypeptide produced in the absence of a G quartet motif sequence.

Preferably the number of G quartet motif sequences is between 1 and 5, more preferably between 3 and 4.

In the use the amount of the polypeptide produced is increased as the number of G quartet motif sequences is increased.

The G quartet motif sequence is suitable for use in an in vitro eukaryote expression system. The G quartet motif sequence is also suitable for use in an in vivo eukaryote expression system.

Preferably in the expression vector the G quartet motif sequence is situated 3′ of the polypeptide open reading frame. When the G quartet motif sequence is incorporated 3′ of the open reading-frame preferably protein production is increased 2-10 fold.

Alternatively in the expression vector the G quartet motif sequence is situated within the polypeptide open reading frame.

Preferably the G-quartet motif encodes the following sequence:

(SEQ ID NO: 4)
NGGN₍₂₋₅₎OGN₍₂₋₅₎GGN₍₂₋₅₎GG

wherein N is any nucleotide.

Further preferably the G quartet motif sequence encodes an RNA consensus sequence of:

(SEQ ID NO: 3)
WGGN₍₁₋₄₎WGGN₍₁₋₄₎WGGN₍₁₋₄₎WGG

wherein N is any nucleotide and W is A or T.
Further preferably the G quartet motif sequence is:

(SEQ ID NO: 2)
GGTATCAGGCAAGGATATGG

Preferably the polypeptide is a human polypeptide. Alternatively the polypeptide is an animal polypeptide. Further preferably the polypeptide is a recombinant polypeptide. Further preferably the polypeptide is a plant polypeptide.

Additionally the eukaryote cell can be further transformed with an expression vector encoding the MID1 protein. Preferably when the host cell is co-transfected with a plasmid expressing the MID1 gene protein production is increased 5-fold. The host cell can also be further transformed with an expression vector encoding the α4 protein.

Preferably the host cell is any insect cell of interest or any plant cell of interest or any mammalian cell of interest or yeast or any transgenic animal cell of interest.

An embodiment is further directed to an expression vector comprising a polynucleotide encoding an open reading frame for a polypeptide and at least one G quartet motif sequence, wherein the G quartet motif sequence increases production of the polypeptide in a eukaryote host cell.

In a host cell transformed with the expression vector the amount of a polypeptide produced in the presence of a G quartet motif sequence is greater than the amount of polypeptide produced in the absence of a G quartet motif sequence.

Preferably the number of G quartet motif sequences is between 1 and 5, more preferably between 3 and 4.

In a host cell transformed with the expression vector the amount of the polypeptide produced is increased as the number of G quartet motif sequences is increased.

The expression vector is suitable for use in an in vitro eukaryote expression system. The expression vector is also suitable for use in an in vivo eukaryote expression system.

Preferably in the expression vector the G quartet motif sequence is situated 3′ of the polypeptide open reading frame. When the G quartet motif sequence is incorporated 3′ of the open reading-frame preferably protein production is increased 2-10 fold.

Alternatively in the expression vector the G quartet motif sequence is situated within the polypeptide open reading frame.

Preferably the G-quartet motif encodes the following sequence:

(SEQ ID NO: 4)
NGGN₍₂₋₅₎GGN₍₂₋₅₎GGN₍₂₋₅₎GG

wherein N is any nucleotide.

Further preferably the G quartet motif sequence encodes an RNA consensus sequence of:

(SEQ ID NO: 3)
WGGN₍₁₋₄₎WGGN₍₁₋₄₎WGGN₍₁₋₄₎WGG

wherein N is any nucleotide and W is A or T.
Further preferably the G quartet motif sequence is:

(SEQ ID NO: 2)
GGTATCAGGCAAGGATATGG

Preferably the polypeptide is a human polypeptide. Alternatively the polypeptide is an animal polypeptide. Further preferably the polypeptide is a recombinant polypeptide. Further preferably the polypeptide is a plant polypeptide.

Additionally the expression vector further comprises polynucleotide sequence encoding the MID1 protein. Preferably when a host cell is co-transfected with an expression vector expressing the MID1 gene protein production is increased 5-fold. The expression vector can also further comprise polynucleotide sequence encoding the α4 protein.

Preferably the host cell is any insect cell of interest or any plant cell of interest or any mammalian cell of interest or yeast or any transgenic animal cell of interest.

### EXAMPLES

### Materials and Methods

### Constructs

pGL3-promoter-, pGL2-promoter- and pRL-CMV vectors were purchased at Promega. In vitro mutagenesis experiments were performed on the pGL3-promoter (primers: F: 5′-CCATCTGCCAGGTATCAGACAAGGATATGGGCTCAC-3′ (SEQ ID NO: 5),

R: GTGAGCCCATATCCTTGTCTGATACCTGGCAGATGG-3′ (SEQ ID NO: 6)) using the QuickChange® Site Directed Mutagenesis Kit (Stratagene) according to manufacturers' instructions.

To produce pGL3m-MIDAS and pGL3m-MIDASm, the following 175 bp DNA-fragment including the MIDAS G-quartet sequence motif (underlined):

(SEQ ID NO: 7)
CACGAAATTGCTTCTGGTGGCGCTCCCCTCTCTAAGGAAGTCGGGGA-
AGCGGTTGCCAAGAGGTTCCATCTGCCAGGTATCAGGCAAGGATATGG
GCTCACTGAGACTACATCAGCTATTCTGATTACACCCGAGGGGGATGA
TAAACCGGGCGCGGTCGGTAAAGTTGTTCCA

or MIDASm (G-nucleotide in italics changed to A):

(SEQ ID NO: 8)
CACGAAATTGCTTCTGGTGGCGCTCCCCTCTCTAAGGAAGTCGGGGA-
AGCGGTTGCCAAGAGGTTCCATCTGCCAGGTATCAGACAAGGATATGG
GCTCACTGAGACTACATCAGCTATTCTGATTACACCCGAGGGGGATGA
TAAACCGGGCGCGGTCGGTAAAGTTGTTCCA

was cloned into the XbaI restriction site of pGL3m

(primers:
(SEQ ID NO: 9)
F: 5′-TGCTCTAGACACGAAATTGCTTCTGGTGGCC-3′,
(SEQ ID NO: 10)
R: 5′-TGCTCTAGATGGAACAACTTTACCGCGCC-3′).

MID1. (wild-type, A130T and C145S) cDNA was excised with HindIII and SalI from pEGFP-C1 and ligated into pCMV-Tag2C (Stratagene).

To create pEGFP-MIDAS, a 60 bp fragment of pGL3 containing the MIDAS G-quartet sequence motif was amplified

| (primers: | |
|---|---|
| (SEQ ID NO: 11) | |
| F: 5′-CCGCTCGAGGCCAAGAGGTTCCATCTGCC-3′, | |
| (SEQ ID NO: 12) | |
| R: 5′-CCAAGCTTTTAGCTGATGTAG-TCTCAGTGAGC-3′ | ) |

and cloned between XhoI and Hind III in the pEGFP-C1 vector (Clontech). GFP-2×MIDAS was created by inserting annealed oligos HE

(HE1:
(SEQ ID NO: 13)
AGCTTGGTATCAGGCAA-GGATATGGG,
HE2:
(SEQ ID NO: 14)
AATTCCCATATCCTTGCCTGATACCA,)

which comprise a core G-quartet sequence with HindIII and EcoRI overhangs into the HindIII and EcoRI restriction sites of GFP-MIDAS. GFP-3MIDAS was created by analogously inserting annealing oligos ES

(ES1:
(SEQ ID NO: 15)
AATTCGGATCAGGCAAGGATATGGG,
ES2:
(SEQ ID NO: 16)
TCGACCCATATCCTTGCCTGATACCG)

with EcoRI and SalI overhangs into the EcoRI and SalI restriction sites of pEGFP-2×MIDAS). To create GFPi-MIDAS, GFP together with the MIDAS G-quartet sequence motif was excised with NheI and EcoRI from GFP-MIDAS and cloned in the MCS of pIRES-DsRed-Express (Clontech).

### RNA Protein Pull Down Assays

Firefly and Renilla cDNA were amplified by PCR with primers including the T7 promoter sequence from the different vectors. In vitro transcriptions were performed using the RiboMAX™ Large scale RNA production system-T7 (Promega), following the manufacturers instructions with some modifications. Briefly, 2-5 μg of purified PCR product were transcribed in the presence of 0.75 mM Biotin-16-uridine-5′-triphosphate (Roche) and 5 mM UTP. Biotinylated RNAs were purified by phenol/chloroform extraction and kept in nuclease free water after ethanol-precipitation.

### RNA-Protein Binding Assay

3×107 HeLa cells were lysed in TKM buffer (20 mM Tris; 50 mM KCl; 5 mM MgCl2) supplemented with proteinase inhibitors (complete mini, Roche), RNase inhibitor (Promega), 1% NP40 and 1 mM DTT on ice, using a 27-G needle. The lysate was cleared by centrifugation for 15 min at 12000 g.

In order to assay proteins for binding to specific RNA, 3 μg of in vitro transcribed and biotinylated RNA were incubated with 200 μg of cytosolic extract of HeLa cells in 500 μl of TKM buffer for 1 hour at 4° C. and, subsequently, with 40 μl of 50% slurry of M-280 streptavidin coated magnetic beads (DYNAL). Beads were washed 3× with TKM buffer for 10 min at 4° C. and boiled off in magic mix (15 mM Tris, 48% Urea, 8.7% glycerol, 1% SDS) for 10 min at 95° C. Bound proteins were analyzed on western blots with anti-FLAG antibody (Stratagene).

### Cell Culture and Transfection

Cos-7, HeLa and HEK293T cells were maintained in DMEM, and CHO cells in DMEM/F12, supplemented with 10% FCS, 2 mM L-glutamine and 100 U/ml penicillin/streptomycin. For FACS analysis, one day before transfection, cells were seeded on six well plates. Next day, Cos-7, HEK and CHO cells were transfected with Lipofectamine™ 2000 (Invitrogen) and HeLa cells with DreamFect™ (OZ biosciences) and 1.5 μs of DNA according to manufactures' instructions. For the luciferase assays, HeLa cells were seeded on 12 well plates and next day transfected with 200 ng Firefly, 10 ng Renilla and 400 ng MID1 construct.

### Luciferase Assay

24 h after translation, HeLa cells were harvested in 1× luciferase passive lysis buffer (Promega). Firefly and Renilla luciferase activities were measured using a Centro LB 960 luminometer (Berthold technologies) and the Dual Luciferase Assay System (Promega) according to manufacturers' instructions. Firefly light units were normalized to Renilla light units.

### In Vitro Transcription/Translation

Specific PCR products of the Firefly luciferase cDNAs were in vitro transcribed with the mMessage mMachine® T7 kit (Ambion). Capped transcripts were in vitro translated with the Flexi® Rabbit reticulocytes lysates (Promega) according to manufactures' instructions. After 90 min Firefly light units were measured using the Luciferase Assay Reagent (Promega) in a luminometer.

### FACS Analysis

24 h after transfection, cells were trypsinized and fixed with ethanol. FACS was analysis was performed on a BD FACSCalibur™. GFP was excited with a 488 nm laser and emission collected using a 530 nm emission filter. A total of 10,000 events/experiment were collected for each construct in three independent experiments. Transfection efficiencies with the different constructs were comparable.

### Quantitative RT-PCR Analysis

RNA from 6 well dishes was isolated using QIAGEN's RNAeasy kit. cDNA was synthesized using the TaqMan reverse transcription reagents kit (Applied Biosystems) with random primers. Primers for real time PCR analysis were designed with the Primer Express 0 Software v2.0

(GFP;
(SEQ ID NO: 17)
F: 5′-CTACCTGAGCACCCAGTCCG-3′;
(SEQ ID NO: 18)
R: 5′-TGATCGCGCTTCTCGTTG-3′; GAPDH).

For real time PCR analysis, absolute quantification with standard curve using SYBRGreen PCR master mix (Applied Biosystems) was performed on an Applied Biosystems ABI Prism 7900HT Sequence Detection System equipped with SDS software v2.

### Example 1

### G-Quartet Motifs Bind to a Microtubule-Associated mRNP

MID1 and α4 are the core of a microtubule-associated mRNP that, in addition to active ribosomes, also assembles G-rich mRNA motifs. In an RNA-protein pull-down experiment, we found that G-quartet structures with the consensus sequence of WGG-N(1-4)-WGG-N(1-4)-WGG-N(1-4)-WGG (SEQ ID NO: 3) have a particularly high affinity to the mRNP.

G-quartet motifs from different mRNAs (EFNBIb, EPBH2b, EFNB2a, EPBH3c) were amplified and in vitro-transcribed with biotinylated ribonucleotides. Transcripts were then immobilized on streptavidin-coated beads and loaded with lysates from MID1-FLAG over-expressing HeLa cells. Elution fractions were analysed on SDS gels using the respective antibodies detecting members of the MID1 protein complex and binding of mRNP to the G-quartet structures was demonstrated.

G-Quartet Motifs Bind to a Microtubule-Associated mRNP in an Additive Manner

A similar protein-RNA pull-down assay with biotinylated RNA in vitro transcribed from a plasmid containing the open reading frame and 3′UTR of ephrin B1 (EFNB1) showed an additive effect of multiple G-quartets on the binding affinity of the respective RNA to the mRNP.

We used EFNB1 mRNAs with differing lengths, including only the open reading frame or the open reading frame and different parts of the 3′UTR with one, two, three or four G-quartets. Antisense transcripts of the two longest mRNAs were used as background controls. G-quartets were amplified and in vitro transcribed using biotinylated ribonucleotides. Elution fractions of RNA-protein pull-down experiments with HeLa cells over-expressing FLAG-tagged MID1 were subsequently analysed by Western blot analysis with an anti-FLAG antibody, and showed a linear increase of the binding affinity between mRNA and MID1 proportional to the number of G-quartet structures (see FIG. 1).

### Example 2

### Comparison of Luciferase Activity: pGL2 and pGL3

Luciferase assays showed a 6-fold higher basal activity of firefly luciferase activity from pGL3 vector compared to the pGL2 vector (see FIG. 2). Ratios between firefly and renilla luciferase were measured in a dual luciferase vector system.

Alignment Between pGL2 and pGL3

Sequence alignments of pGL2 and pGL3 show identity of the two vectors over large parts of the sequence. Only a few base pair mismatches were detected within the open reading frame of the firefly luciferase. Among these, four base pair exchanges were found to be located in a G-quartet structure predicted for pGL3, thus eliminating the motif in pGL2.

pGL2
(SEQ ID NO: 1)
GGGATACGACAAGGATATGG
pGL3
(SEQ ID NO: 2)
GGTATCAGGCAAGGATATGG

### Example 3

### Translation Decrease in a pGL3 Vector with Mutated G-Quartet

Next, we mutated one of the core guanines of the G-quartet predicted in the pGL3 firefly luciferase gene such that the amino acid sequence did not change (core guanine at position 9 of the pGL3 motif sequence shown above was mutated to adenine), and checked luciferase activity of the mutated vector pGL3mut. Interestingly, basal activity of the mutated vector went down significantly (see FIG. 3).

### Example 4

### Increase of Translation by Co-Transfection with MID1

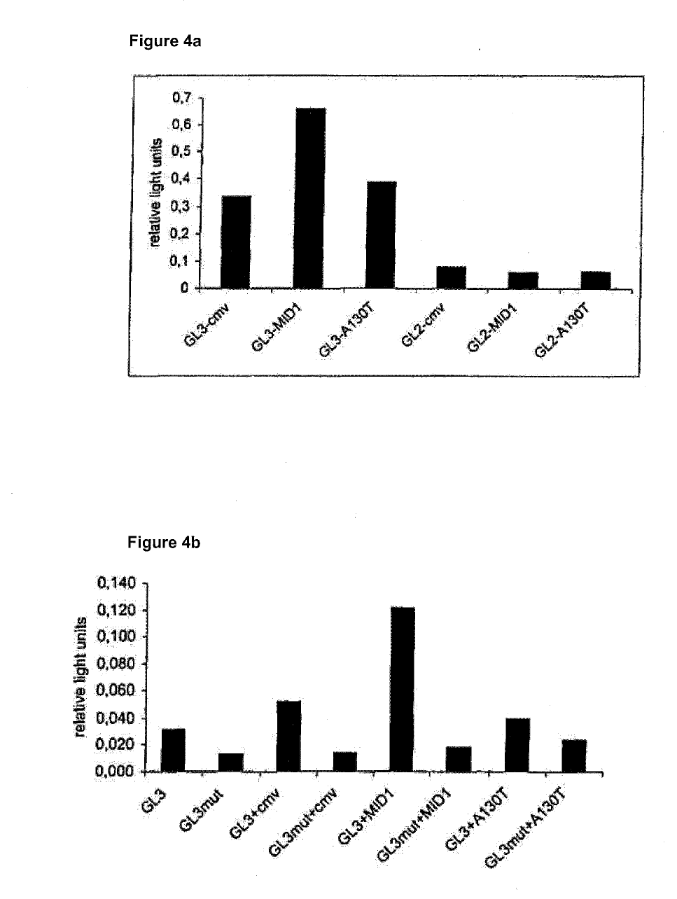

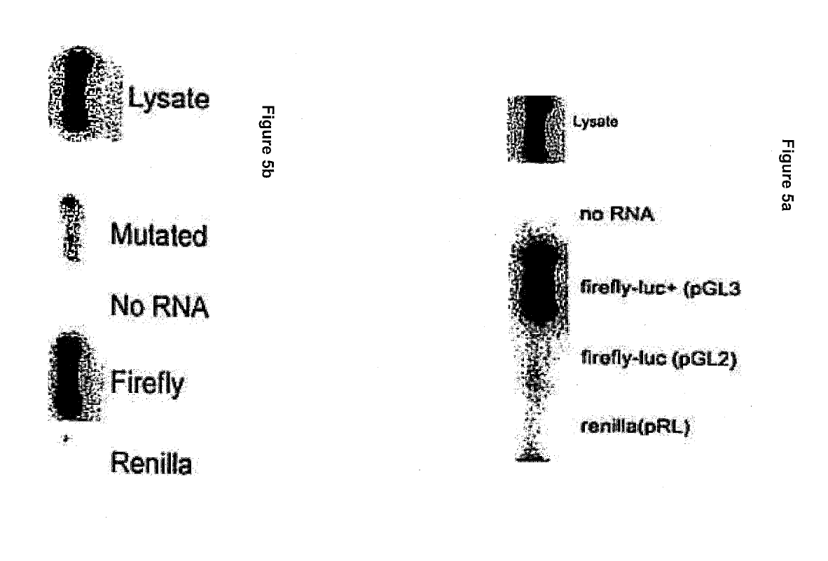

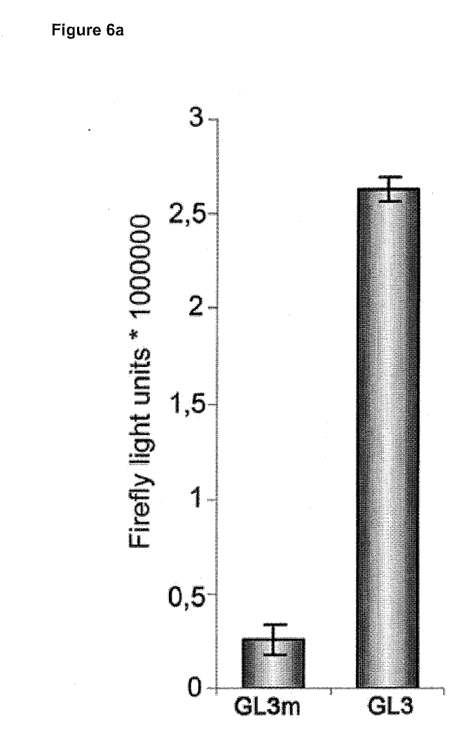

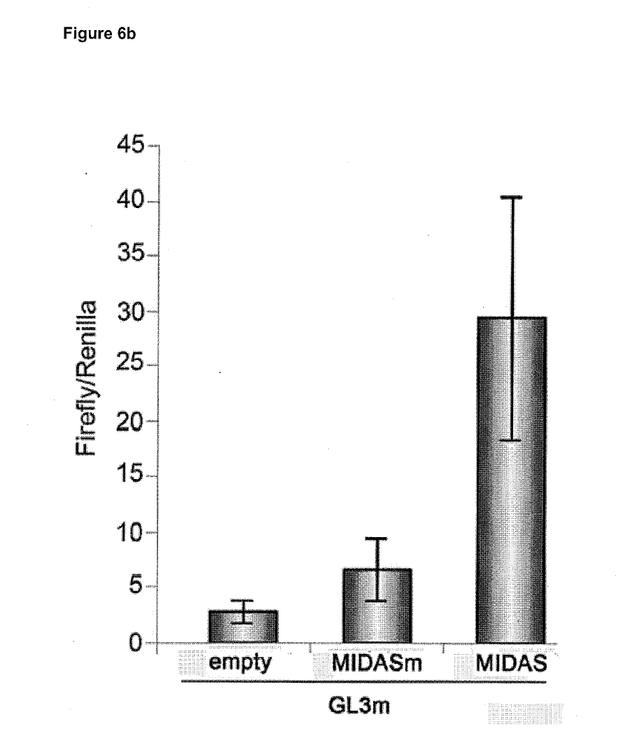

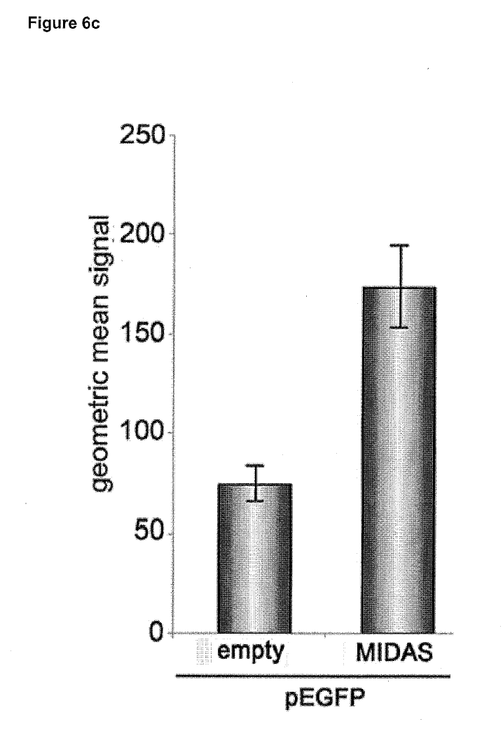

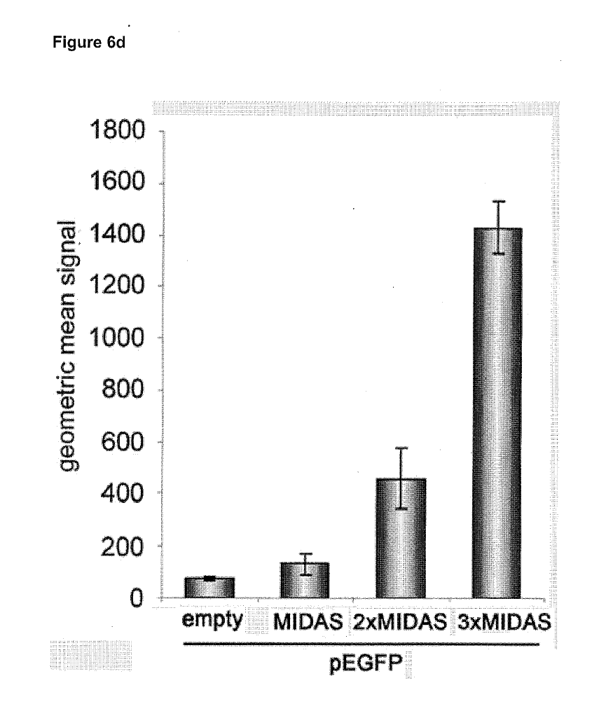

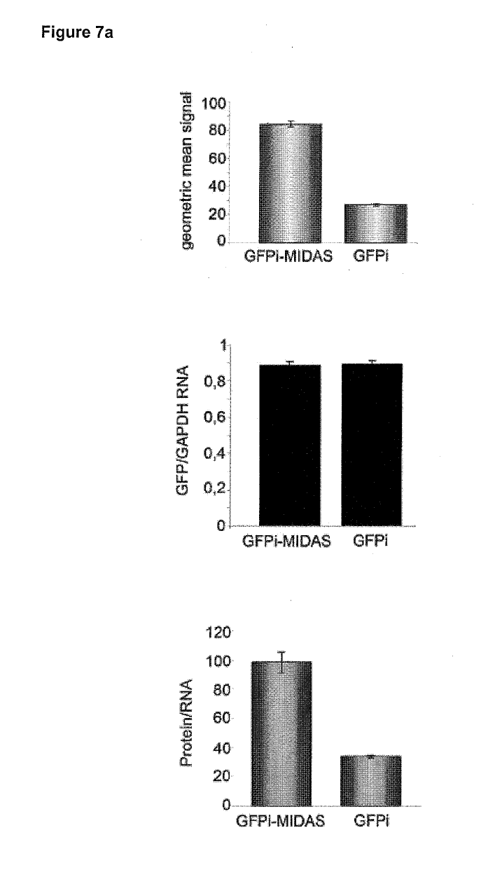

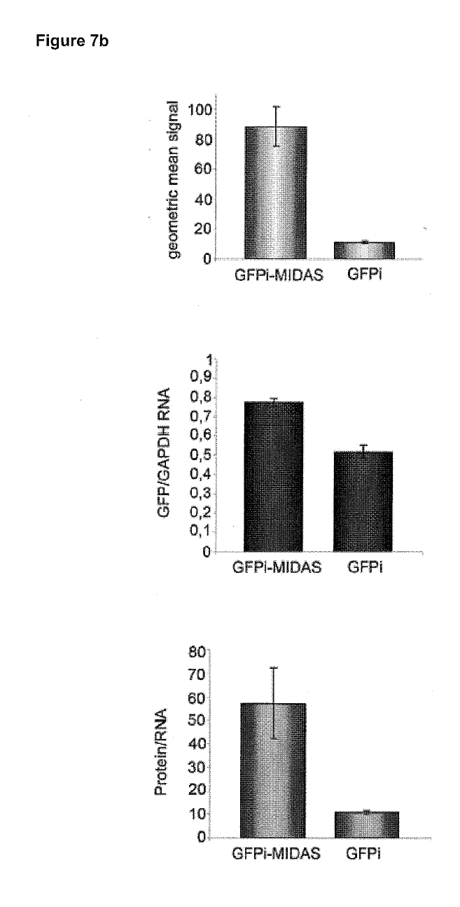

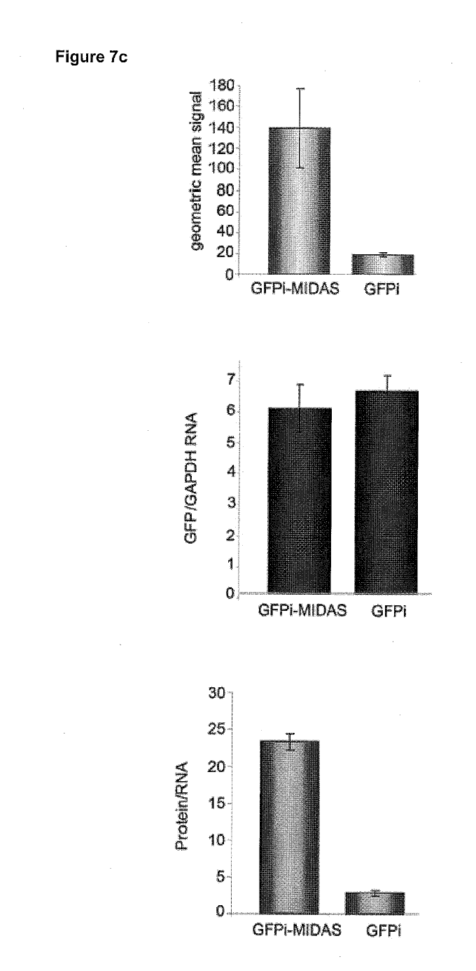

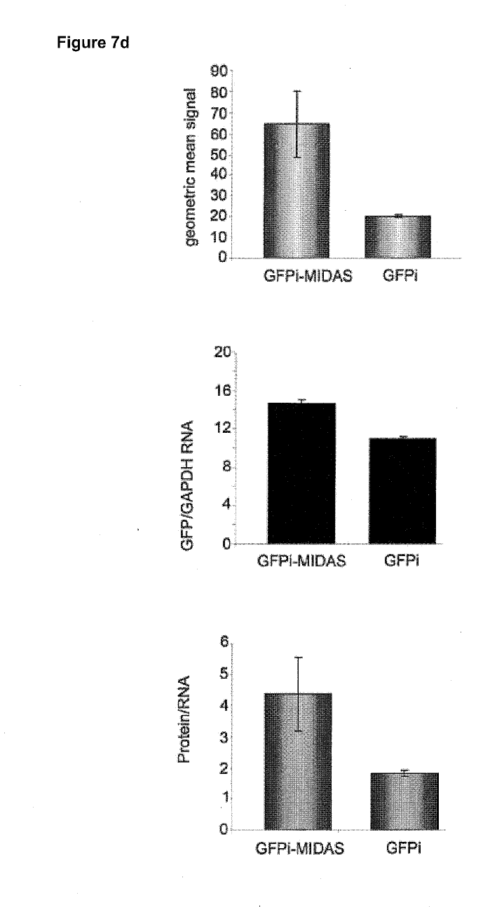

To test the hypothesis that the MID1 complex modulates the efficiency of protein production from associated mRNAs, we overexpressed either a FLAG-MID1 wt construct or a FLAG-MID1 construct containing inactive MID1 or an empty pCMV vector in HeLa cells and analyzed its influence on the expression of firefly luciferase using a dual luciferase reporter assay. For this assay FLAG-MID1 or pCMV expressing cells were co-transfected with a renilla luciferase vector and one or the other of two different firefly luciferase vectors (firefly pGL3-promoter or firefly pGL2-promoter, respectively). Unexpectedly, we observed a >2-fold MID1-dependent induction of firefly luciferase activity compared with that seen following co-expression with an empty pCMV vector or with a pCMV construct that encodes an inactive MID1 protein, but only in the presence of the pGL3, not the pGL2 vector (FIG. 4a). Comparison of the pGL2 and pGL3 vector sequences indicated that all but a few nucleotides within the open reading frame of the firefly luciferase genes were identical, and that some of the changed nucleotides in pGL3 reside within the MIDAS signature sequence of a G-quartet like structure, a putative protein-RNA binding motif, whereas in pGL2 this MIDAS G-quartet like motif is destroyed (see pGL2 and pGL2 motif sequences in Example 2 above).

To determine whether the MIDAS G-quartet like structure of pGL3 is required for its ability to enhance pGL3 firefly luciferase activity, a core guanine at position 9 of the pGL3 motif sequence shown in Example 2 above was mutated to adenine. This left the amino acid sequence of the firefly luciferase intact, and both the mutated and non-mutated codons are common in eukaryotic cells. It was found that transfection with the mutated vector virtually eliminated the induction of luciferase activity caused by co-expression with wild-type MID1, as occurred with the wild-type pGL3 vector. In addition, basal firefly luciferase activity was decreased upon transfection with the mutated pGL3 vector, probably owing to the loss of the positive regulatory influence of endogenous MID1 complex upon translation of the mutated mRNA (FIG. 4b).

Thus co-transfection of dual luciferase vectors (firefly, pGL and, as an internal control, renilla, pRL) with an expression vector containing the MID1 open reading frame showed an upregulation of firefly luciferase activity only when using the wild-type pGL3 vector, but not with the mutated pGL3 vector carrying a single substitution mutation in the G-quartet (FIG. 4b) or the pGL2 vector (FIG. 4a). Co-expression of the two luciferase vectors with a MID1-containing plasmid carrying a missense mutation that leads to dysfunction of the MID1 protein did not result in any induction of luciferase activity. Ratios between firefly and renilla luciferase were measured in a dual luciferase reporter system.

### Example 5

### Binding of the pGL3 Firefly Luciferase mRNA to the MID1 Protein Complex

It was found that FLAG-MID1 bound specifically to biotin-labeled luciferase mRNA in vitro-transcribed from the pGL3-firefly luciferase vector, but not to mRNAs transcribed from pGL2- or pRL-renilla luciferase vectors, using a RNA-protein pull-down assay (FIG. 5a). Specificity of the reaction was verified by omission of RNA from the assay (FIG. 5a). From this data we conclude that, while the MID1 protein binds to the firefly luciferase mRNA derived from the pGL3 vector thereby enhancing pGL3-firefly luciferase activity, there is no binding between the MID1 protein and the pGL2 luciferase mRNA and consequently also no influence of MID1 on the pGL2 luciferase activity.

Also the ability of the mutated pGL3 (having a core guanine at position 9 of the pGL3 motif sequence shown in Example 2 above mutated to adenine) firefly luciferase mRNA to bind to FLAG-MID1 was significantly reduced as compared with that of wild-type mRNA, as shown by RNA-protein pull-down assay (FIG. 5b). Again, renilla mRNA and an experiment without RNA served as background controls.

Thus, specific binding of the MID1-protein complex to the wildtype pGL3 firefly luciferase mRNA (FIGS. 5a and 5b) but not to the pGL2 firefly luciferase mRNA (FIG. 5a) nor to a pGL3 firefly luciferase mRNA carrying a single substitution mutation in the G-quartet (FIG. 5b) could be shown in an RNA-protein pulldown assay with biotin-streptavidin immobilized RNA and cell lysate from HeLa cells overexpressing FLAG-MID1. Elution fractions were analysed on a Western blot with an anti-FLAG antibody.

### Example 6

The observed increase in protein production driven by the MIDAS G-quartet sequence motif could either result from increased transcription, RNA stability, and/or translation efficiency. To distinguish among these, in vitro transcription/translation from the wild-type and MIDAS-mutated forms of pGL3 was performed. Using rabbit reticulocyte lysates, which contain substantial endogenous MID1 levels (data not shown) a 10-fold increase in protein translation was observed from the RNA containing the intact MIDAS motif vs. its single-site substitution mutated form (FIG. 6a), supporting a major impact of the MIDAS motif on translation efficiency.

We also asked whether the position of the MIDAS G-quartet sequence motif within an mRNA alters its translation-promoting activity. Transferability of the MIDAS motif and its translation-enhancing properties from the open reading frame into the 3′ or 5′ untranslated regions of mRNAs would be of great value for biotechnological protein production processes. Also, because biotechnological protein production heavily depends on optimal viability of the protein-producing cells, and overexpression of MID1 has a negative effect on cell viability (unpublished data), we decided to study putative MIDAS G-quartet sequence motif dependent enhancements of protein synthesis, which we designated the “MIDAS effect” without ectopic expression of MID1, instead relying upon the activity of the endogenous complex. Endogenous MID1 and its protein interaction partners are ubiquitously expressed in many tissues and cell lines. Intact or single substitution mutated (G to A) MIDAS motifs were cloned 3′ to the luciferase coding sequence of a pGL3 vector whose intrinsic MIDAS motif contained an inactivating mutation (pGL3mut). These constructs were transfected into CHO cells, which are commonly used in biotechnological processes, and the resulting luciferase activity measured. The results show a several-fold increase of luciferase activity after transfection of the pGL3mut vector containing a functional MIDAS motif 3′ to the luciferase coding sequence, as compared with that seen with a vector carrying a non-functional motif (FIG. 6b).

Furthermore, it was found that a strong MIDAS effect also resulted when the motif was cloned 3′ to the coding region of the green fluorescent protein (GFP) gene in a pEGFP-C1 vector (FIG. 6c). Together, these results show that the MIDAS motif can increase cellular production of proteins encoded by transfected genes, independent of (i) its position within the mRNA, (ii) the particular protein produced, and (iii) the vector used for protein expression.

Furthermore, we asked whether the MIDAS effect is effectively dose-dependent, such that it can be potentiated by inclusion of multiple MIDAS G-quartet sequence motifs. To test this, we introduced one and two additional MIDAS sequences into the 3′UTR of the GFP-MIDAS vector. Transfection of CHO cells with these constructs containing multiple MIDAS motifs in the 3′UTR of the GFP gene enhanced translation efficiency up to 20 fold in CHO cells (FIG. 6d). Thus, increasing the number of MIDAS motifs can lead to an additive increase in translation efficiency, which seems to be based on additive binding of MID1 to the respective RNA (unpublished results).

### Example 7

To determine whether the enhancement of protein production by MIDAS G-quartet sequence motif is cell type-dependent, and also the relative importance of the MIDAS motifs influence upon mRNA stability and protein translation, we studied the MIDAS effect in the GFP-IRES-vector system in four different cell lines and measured simultaneously the fluorescence intensity of the protein produced, by FACS (top panels of FIGS. 7a, 7b, 7c and 7d), and the corresponding GFP mRNA levels relative to those of GAPDH, by real-time PCR (centre panels of FIGS. 7a, 7b, 7c and 7d). A MIDAS motif was cloned 3′ to the coding region for the GFP protein, and a vector lacking the MIDAS motif was used as control. Strong MIDAS effects upon production of GFP was seen in all cell lines tested (top panels of FIG. 7a-HeLa, FIG. 7b-CHO, FIG. 7c-COST and FIG. 7d-HEK293), while its influence on the mRNA levels seemed to play a minor role in the overall effect (centre panels of FIGS. 7a, 7b, 7c and 7d). Nevertheless, the strongest MIDAS effect, seen in HEK293 cells (8-fold enhancement) was accompanied by a substantial (50%) increase in GFP mRNA relative to that of GAPDH (centre panels of FIG. 7b), suggesting a synergistic effect potentially involving increased mRNA stability as well as translation. The translation efficiency was measured by the ratios between fluorescence intensities and mRNA expression (bottom panels of FIGS. 7a, 7b, 7c and 7d).

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A method for increasing production of a polypeptide in a eukaryote host cell comprising:
a) transforming the eukaryote host cell with an expression vector comprising a polynucleotide sequence that encodes an open reading frame for the polypeptide and at least one G quartet motif sequence;
b) culturing the transformed cells;
c) isolating the expressed polypeptide.

2. The method of claim 1, wherein the number of G quartet motif sequences is between 1 and 5.

3. The method of claim 1, wherein the amount of the polypeptide produced is increased as the number of G quartet motif sequences is increased

4. The method of claim 1, wherein in the expression vector the G quartet motif sequence is situated 3′ of the polypeptide open reading frame.

5. The method of claim 1, wherein in the expression vector the G quartet motif sequence is situated within the polypeptide open reading frame.

6. The method of claim 1, wherein the G quartet motif sequence encodes an RNA consensus sequence of:
(SEQ ID NO: 3)
WGGN₍₁₋₄₎WGGN₍₁₋₄₎WGGN₍₁₋₄₎WGG.

7. The method of claim 1, wherein the polypeptide is a human polypeptide.

8. The method of claim 1, wherein the polypeptide is an animal polypeptide.

9. The method of claim 1, wherein the polypeptide is a plant polypeptide.

10. The method of claim 1, wherein the polypeptide is a recombinant polypeptide.

11. The method of claim 1, wherein the eukaryote cell is further transformed with an expression vector encoding the MID1 protein.

12. The method of claim 1, wherein the eukaryote cell is further transformed with an expression vector encoding the α4 protein.

13. Use of a G quartet motif sequence to increase production of a polypeptide in a eukaryote host cell, wherein the eukaryote cell is transformed with an expression vector comprising a polynucleotide sequence that encodes an open reading frame for the polypeptide and at least one G quartet motif sequence.

14. An expression vector comprising a polynucleotide encoding an open reading frame for a polypeptide and at least one G quartet motif sequence, wherein the G quartet motif sequence increases production of the polypeptide in a eukaryote host cell.
